(19) United States
(12) Patent Application Publication
(10) Pub. No.: US 2013/0019338 A1
(43) Pub. Date: Jan. 17, 2013

(54) CORN PRODUCTS AND METHODS FOR THEIR PRODUCTION

(71) Applicant: Steve J. Plehn, Juneau, WI (US)

(72) Inventor: Steve J. Plehn, Juneau, WI (US)

(73) Assignee: AGRIGENETICS, INC., Indianapolis, IN (US)

(21) Appl. No.: 13/549,256

(22) Filed: Jul. 13, 2012

Related U.S. Application Data
(60) Provisional application No. 61/507,624, filed on Jul. 14, 2011.

(51) Int. Cl. A01H 5/00; A01H 1/02; A01H 5/06; A01H 5/02; A01H 5/12; C12N 5/04; A01H 5/10

(52) U.S. Cl. 800/275; 435/412; 800/303; 800/320.1

(57) ABSTRACT

The present invention relates to inbred corn plants and seed as well as hybrid corn plants and seed comprising both a brown-midrib and a floury-endosperm genotype.

### CROSS-REFERENCE TO RELATED APPLICATION

This application claims the benefit of U.S. Provisional Patent Application Ser. No. 61/507,624, filed Jul. 14, 2011, the disclosure of which is hereby incorporated herein in its entirety by this reference.

### FIELD OF THE INVENTION

The present invention relates to inbred corn plants and seed as well as hybrid corn plants and seed comprising both a brown-midrib and a floury-endosperm genotype.

### BACKGROUND OF THE INVENTION

Corn plants (Zea mays L.) can be bred by both self-pollination and cross-pollination. Both types of pollination involve the corn plant's flowers. Corn has separate male and female flowers on the same plant, located on the tassel and the ear, respectively. Natural pollination occurs in corn when wind blows pollen from the tassels to the silks that protrude from the tops of the ear shoot. Breeding techniques take advantage of a plant's method of pollination. Thus, by controlling the pollination process, plant breeding allows to production progeny specifically from selected parent plants.

North American farmers plant tens of millions of acres of corn at the present time and there are extensive national and international commercial corn breeding programs. A variety of naturally occurring mutations are known for various corn varieties, but traits that are agronomically advantageous are often accompanied by other undesirable characteristics. One goal of corn plant breeding, therefore, is the introgression of advantageous genes into an agronomically superior genetic background to produce plants that of greater commercial value.

The COMT gene encodes caffeic acid O-methyltransferase, which is involved in lignin biosynthesis. Brown-midrib-3 (bm3) mutations in the COMT gene cause a decrease in the lignin content in roots, stems, and leaves of corn plants, and cause a reddish-brown pigmentation in the leaf midrib. Decreased lignin is a desirable trait in corn crops used for fodder because it increases the digestibility of that fodder when fed to livestock.

Zeins are prolamin storage proteins in the endosperm of corn seeds. The floury-2 (fl2) allele in corn causes a decrease in the synthesis of zein proteins resulting in a floury endosperm, which is another desirable trait in animal feed because of increased digestibility. Floury endosperm is digested more rapidly and completely than vitreous endosperm.

The genes for bm3 and fl2 are tightly linked approximately 5 cM genetic-distance apart on maize chromosome 4, with the bm3 and fl2 alleles in trans linkage disequilibrium among corn germplasm. Meiotic crossing over between these two loci is rare. The particular recessive alleles of bm3 and fl2 have not been previously fixed in a homozygous cis configuration in one genotype, nor have they been dispersed together in this cis configuration into breeding lines to cross together to produce corn hybrids that are homozygous for these alleles and thereby express both the brown-midrib and floury-endosperm traits. Because corn germplasm has strong linkage disequilibrium between these two closely linked recessive alleles, corn seed comprising both a brown-midrib and a floury-endosperm genotype has heretofore been unknown.

### DISCLOSURE OF THE INVENTION

In the description and examples that follow, a number of terms are used. To provide a clear and consistent understanding of the specification and claims, including the scope to be given such terms, the following definitions are provided.

Anther Color: Recorded at the time of pollen shed when anthers are actively dehiscing pollen as a standard color name [Light Green (1), Green-Yellow (5), Pale Yellow (6), Yellow (7), Salmon (9), Pink (11), Cherry Red (13), Purple (17), Tan (22)] and Munsell color code.

Brown midrib: The recessive bm3 allele, located on the short arm of chromosome 4, gives plants a reddish-brown pigment in the leaf mid-vein starting when there are four to six leaves. In addition, it affects the activity of catechol O-methyl transferase to decrease lignin concentration, which improves forage digestibility for ruminants.

Digestibility: Percentage of whole silage (ensiled stover and grain) or feed-ration components that is digested by animals. Greater digestibility is associated with higher energy intake.

Endosperm Type: Region of the kernel between the germ and the seed coat; rated as sweet, extra sweet (sh2), normal starch, high amylase starch, waxy, high protein, high lysine, supersweet (se), high oil and other-specify.

Floury endosperm: Characterized by lower prolamin content and less starch encapsulation, giving the endosperm a soft, chalky texture and opaque appearance.

Glume Color: Color of the glume after exposure to sunlight and just before extruding anthers; recorded as a standard color name [Light Green (1), Medium Green (2), Dark Green (3), Very Dark Green (4), Green-Yellow (5), Salmon (9), Pink (11), Cherry Red (13), Red (14), Pale Purple (16)] and Munsell color code.

Grain Light Transmission: Relative amount of light that will pass through a corn kernel.

NDF (Neutral Detergent Fiber): Hemicellulose, cellulose, lignin, and cutin (plant structural material) as a percentage of the whole plant on a dry-matter basis after digestion in a non-acidic, non-alkaline detergent.

NDFD: Percentage of neutral detergent fiber that is digestible; determined in vitro by incubating a ground feed sample in live rumen fluid and measuring its disappearance to simulate the amount and rate of digestion that would occur in the rumen.

Plant Height: Plant height in centimeters from the ground to the tip of the tassel.

Silk Color: Color of the silk three days after its emergence; recorded as standard color name [Light Green (1), Green-Yellow (5), Pale Yellow (6), Yellow (7), Salmon (9), Pink-Orange (10), Pink (11), Cherry Red (13), Purple (17), Tan (22)] and Munsell color code.

Tillers: Branches that develop from axillary buds at the lower five to seven stalk nodes of a corn plant; they are morphologically identical to the main stalk and capable of forming their own root system, nodes, internodes, leaves, ears, and tassels.

True Breeding: A line is considered true breeding for a particular trait if it is genetically homozygous for that trait to the extent that when the variety is self-pollinated, no significant amount of independent segregation of the trait among progeny is observed.

An object of the present invention is a corn seed comprising a homozygous bm3 and fl2 genotype and a brown-midrib and a floury-endosperm phenotype.

Another object of the present invention is seed of a corn inbred line comprising a homozygous bm3 and fl2 genotype and a brown-midrib and a floury-endosperm phenotype, or a part thereof.

A further object of the present invention is a hybrid corn seed comprising a homozygous bm3 and fl2 genotype and a brown-midrib and a floury-endosperm phenotype.

Additional objects and advantages of the present invention will become readily apparent to those skilled in this art from the following detailed description, wherein embodiments of the invention are described simply by way of illustrating the best mode contemplated in carrying out the invention. As will be realized, the invention is capable of other and different embodiments, and its several details are capable of modifications in various obvious respects, all without departing from the invention. Accordingly, the description is to be regarded as illustrative in nature and not as restrictive.

### DETAILED DESCRIPTION OF THE INVENTION

The present inventions will be described more fully hereinafter. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

In accordance with one aspect of the present invention, provided is an inbred corn seed and plants thereof exhibiting a bm3 and fl2 genotype and a brown-midrib and floury-endosperm phenotype. The present invention further relates to a method for producing inbred corn seeds that includes, but is not limited to, the steps of planting seed of the inventive corn in proximity to itself, growing the resulting corn plants under self-pollinating conditions with adequate isolation, and harvesting resultant seed obtained from such inbred plants using techniques standard in the agricultural arts such as would be necessary to bulk-up seed such as for hybrid production. The present invention also relates to inbred seed produced by such a method.

The present invention also relates to one or more plant parts of a corn plant exhibiting a brown-midrib genotype and a floury-endosperm genotype. Corn plant parts include plant cells, plant protoplasts, plant cell tissue cultures from which corn plants can be regenerated, plant calli, plant clumps, and plant cells that are intact in plants or parts of plants, such as embryos, pollen, ovules, flowers, seeds, kernels, ears, cobs, leaves, husks, stalks, roots, root tips, brace roots, lateral tassel branches, anthers, tassels, glumes, silks, tillers, and the like.

In another aspect of the present invention, generally referred to as backcrossing, the brown-midrib and floury-endosperm traits may be introduced into an inbred parent corn plant (the recurrent parent) by crossing the inbred corn plants with another corn plant (referred to as the donor or non-recurrent parent) which carries the gene(s) encoding the particular brown-midrib and floury-endosperm trait(s) of interest to produce F₁ progeny plants. Both dominant and recessive alleles may be transferred by backcrossing. The donor plant may also be an inbred, but in the broadest sense can be a member of any plant variety or population cross-fertile with the recurrent parent. Next, F₁ progeny plants that have the desired trait are selected. Then, the selected progeny plants are crossed with the inbred parent plant to produce backcross progeny plants. Thereafter, backcross progeny plants comprising both the desired brown-midrib and floury-endosperm traits and the physiological and morphological characteristics of the inbred corn plant are selected. This cycle is repeated for about one to about eight cycles, preferably for about 3 or more times in succession to produce selected higher backcross progeny plants that comprise the desired trait and all of the physiological and morphological characteristics of corn inbred line as determined at the 5% significance level when grown in the same environmental conditions. One of ordinary skill in the art of plant breeding would appreciate that a breeder uses various methods to help determine which plants should be selected from the segregating populations and ultimately which inbred lines will be used to develop hybrids for commercialization. In addition to the knowledge of the germplasm and other skills the breeder uses, a part of the selection process is dependent on experimental design coupled with the use of statistical analysis. Experimental design and statistical analysis are used to help determine which plants, which family of plants, and finally which inbred lines and hybrid combinations are significantly better or different for one or more traits of interest. Experimental design methods are used to assess error so that differences between two inbred lines or two hybrid lines can be more accurately determined. Statistical analysis includes the calculation of mean values, determination of the statistical significance of the sources of variation, and the calculation of the appropriate variance components. Either a five or a one percent significance level is customarily used to determine whether a difference that occurs for a given trait is real or due to the environment or experimental error. One of ordinary skill in the art of plant breeding would know how to evaluate the traits of two plant varieties to determine if there is no significant difference between the two traits expressed by those varieties. For example, see Fehr, Walt, Principles of Cultivar Development, p. 261-286 (1987) which is incorporated herein by reference. Mean trait values may be used to determine whether trait differences are significant, and preferably the traits are measured on plants grown under the same environmental conditions.

This method results in the generation of inbred corn plants with substantially all of the desired morphological and physiological characteristics of the recurrent parent and the particular transferred trait(s) of interest. Because such inbred corn plants are heterozygous for loci controlling the transferred trait(s) of interest, the last backcross generation would subsequently be selfed to provide true breeding progeny for the transferred trait(s).

Backcrossing may be accelerated by the use of genetic markers such as SSR, RFLP, SNP, AFLP, or other markers to identify plants with the greatest genetic complement from the recurrent parent. In yet another aspect of the invention, processes are provided for producing corn seeds or plants, which processes generally comprise crossing a first parent corn plant with a second parent corn plant wherein the first parent corn plant and the second parent corn plant are both inbred corn plants exhibiting a bm3 and fl2 genotype and a brown-midrib and floury-endosperm phenotype.

Any time two different inbred corn plants according to the present invention are crossed with one another, a first generation (F₁) corn hybrid plant is produced. As such, any F₁ hybrid corn plant or corn seed exhibiting both a bm3 and fl2 genotype and a brown-midrib and floury-endosperm phenotype are part of the present invention.

When an inbred corn plant exhibiting both a bm3 and fl2 genotype and a brown-midrib and floury-endosperm phenotype is crossed with another inbred plant exhibiting both a bm3 and fl2 genotype and a brown-midrib and floury-endosperm phenotype to yield a hybrid exhibiting both a bm3 and fl2 genotype and a brown-midrib and floury-endosperm phenotype, the original inbreds can serve as either the maternal or paternal plant with basically, the same characteristics in the hybrids. Occasionally, maternally inherited characteristics may express differently depending on the decision of which parent to use as the female. However, often one of the parental plants is preferred as the maternal plant because of increased seed yield and preferred production characteristics, such as optimal seed size and quality or ease of tassel removal. Some plants produce tighter ear husks leading to more loss, for example due to rot, or the ear husk may be so tight that the silk cannot completely push out of the tip preventing complete pollination resulting in lower seed yields. There can be delays in silk formation which deleteriously affect timing of the reproductive cycle for a pair of parental inbreds. Seed coat characteristics can be preferable in one plant which may affect shelf life of the hybrid seed product. Pollen can shed better by one plant, thus rendering that plant as the preferred male parent.

In embodiments of the present invention, the first step of “crossing” the first and the second parent corn plants comprises planting, preferably in pollinating proximity, seeds of a first inbred corn plant and a second, distinct inbred corn plant. The seeds of the first inbred corn plant and/or the second inbred corn plant can be treated with compositions that render the seeds and seedlings grown therefrom more hardy when exposed to adverse conditions.

A further step comprises cultivating or growing the seeds of the first and second parent corn plants into plants that bear flowers. If the parental plants differ in timing of sexual maturity, techniques may be employed to obtain an appropriate nick, i.e., to ensure the availability of pollen from the parent corn plant designated the male during the time at which silks on the parent corn plant designated the female are receptive to the pollen. Methods that may be employed to obtain the desired nick include delaying the flowering of the faster maturing plant, such as, but not limited to delaying the planting of the faster maturing seed, cutting or burning the top leaves of the faster maturing plant (without killing the plant) or speeding up the flowering of the slower maturing plant, such as by covering the slower maturing plant with film designed to speed germination and growth or by cutting the tip of a young ear shoot to expose silk.

In a preferred embodiment, the corn plants are treated with one or more agricultural chemicals as considered appropriate by the grower.

A subsequent step comprises preventing self-pollination or sib-pollination of the plants, i.e., preventing the silks of a plant from being fertilized by any plant of the same variety, including the same plant. This is preferably done in large scale production by controlling the male fertility, e.g., treating the flowers so as to prevent pollen production or alternatively, using as the female parent a male sterile plant of the first or second parent corn plant (i.e., treating or manipulating the flowers so as to prevent pollen production, to produce an emasculated parent corn plant or using as a female, a cytoplasmic male sterile version of the corn plant). This control may also be accomplished in large scale production by physical removal of the tassel from the female plant, either by pulling the tassel by hand, cutting with a rotary cutter, or pulling with a mechanical tassel pulling machine. In small scale production, corn breeder's shoot bags, usually plastic or glassine, applied to cover the ear shoot prior to the extrusion of silks provide effective control of unwanted self-pollination or sib-pollination.

Yet another step comprises allowing cross-pollination to occur between the first and second parent corn plants. When the plants are not in pollinating proximity, this is done by placing a bag, usually paper, over the tassels of the first plant and another shoot bag over the ear shoot, prior to the extrusion of silk, of the incipient ear on the second plant. The bags are left in place usually overnight. Since pollen stops shedding each day and loses viability and new pollen is shed each morning, this assures that the silks are not pollinated from other pollen sources, that any stray pollen on the tassels of the first plant is dead, and that the only pollen transferred comes from the first plant. The pollen bag over the tassel of the first plant is then shaken vigorously to enhance release of pollen from the tassels and removed from the first plant. Finally, in one continuous motion, the shoot bag is removed from the silks of the incipient ear on the second plant, and the pollen bag containing the captured pollen is placed over the silks of the incipient ear of the second plant, shaken again to disperse the captured pollen, and left in place covering the developing ear to prevent contamination from any unwanted fresh airborne pollen. In large scale production, crossing is accomplished by isolated open-pollinated crossing fields whereby corn plants of the parent designated as the female, which are controlled for male fertility, are allowed to be pollinated by other plants of a different corn type where such plants are adjacent to the plants designated as the female parent.

A further step comprises harvesting the seeds, near or at maturity, from the ear of the plant that received the pollen. In a particular embodiment, seed is harvested from the female parent plant, and when desired, the harvested seed can be grown to produce a first generation (F₁) hybrid corn plant exhibiting both a brown-midrib genotype and a floury-endosperm genotype.

Yet another step comprises drying and conditioning the seeds, including the treating, sizing (or grading) of seeds, and packaging for sale to growers for the production of grain or forage. As with inbred seed, it may be desirable to treat hybrid seeds with compositions that render the seeds and seedlings grown therefrom more hardy when exposed to adverse conditions. Mention should be made that resulting hybrid seed is sold to growers for the production of grain and forage and not for breeding or seed production.

A single-cross hybrid is produced when two different inbred parent corn plants are crossed to produce first generation F₁ hybrid progeny. Generally, each inbred parent corn plant has a genotype which complements the genotype of the other inbred parent. Typically, the F₁ progeny are more vigorous then the respective inbred parent corn plants. This hybrid vigor, or heterosis, is manifested in many polygenic traits, including markedly improved yields and improved stalks, roots, uniformity and insect and disease resistance. It is for this reason that single cross F1 hybrids are generally the most sought after hybrid.

### EXAMPLES

The following example is included to demonstrate certain preferred embodiments of the invention. This example should not be construed as limitations to the claims. It should be appreciated by those of skill in the art that the techniques disclosed in the following example represents specific approaches used to illustrate preferred modes for its practice. However, those of skill in the art should, in light of the present disclosure, appreciate that many changes can be made in these specific embodiments while still obtaining like or similar results without departing from the spirit and scope of the invention.

In a preferred embodiment, the inbred corn seed and plants thereof are seed and plants of inbred corn line 09SMA31BF. A description of physiological and morphological characteristics, including those relating to the bm3 and fl2 genotype, of corn plant 09SMA31BF is presented in Table 1.

TABLE 1
Physiological and Morphological Characteristics of 09SMA31BF

| Characteristic | Value |
|---|---|
| leaf mid-rib color V4 to V6^{a} | reddish brown |
| stalk color^{a} | reddish brown |
| grain light transmision^{b} | opaque |
| Anther Color (standard) | Pale Yellow |
| Glume Color (standard) | Medium Green |
| Silk Color (standard) | Pink |
| Plant Height (cm) | 230 |
| Tillers Present (Y/N) | No |
| Anthocyanin in brace roots | Dark |
| Leaf Color (standard) | Dark Green |
| Upper Leaf Angle | Intermediate |
| Ear Node Leaf Width (cm) | 9.2 |
| Leaf Margin Color | White |
| Lateral Tassel Branches (count) | 9.5 |
| Ears Per Stalk (count) | 1 |
| Ear Length (cm) | 15.25 |
| Number of Kernel Rows (count) | 14 |
| Kernel Row Alignment (description) | Slightly Curved |
| Ear Taper (1 = slight, 2 = avg, 3 = extreme) | 2 |
| Cob Color (standard) | Red |
| Endosperm Type | Normal Starch |

^{a}Characteristic of the homozygous bm3 genotype.
^{b}Characteristic of the homozygous fl2 genotype.

It should be appreciated by one having ordinary skill in the art that, for the quantitative characteristics identified in Table 1, the values presented are typical values. These values may vary due to the environment and accordingly, other values that are substantially equivalent are also within the scope of the invention.

Inbred corn line 09SMA31BF shows uniformity and stability within the limits of environmental influence for the traits described in Table 1. Inbred 09SMA31BF has been self-pollinated and ear-rowed a sufficient number of generations with careful attention paid to uniformity of plant type to ensure the homozygosity and phenotypic stability necessary to use in large scale, commercial production. The line has been increased both by hand and sib-pollinated in isolated fields with continued observations for uniformity. No variant traits have been observed or are expected in 09SMA31BF.

Applicants have made a deposit of at least 2,500 seeds of inbred corn plant 09SMA31BF with the American Type Culture Collection (ATCC), Manassas, Va. 20110 USA, under ATCC Accession No. ______. The seeds deposited with the ATCC on ______ were taken from a deposit maintained by Agrigenetics, Inc. d/b/a Mycogen Seeds since prior to the filing date of this application. Access to this deposit will be available during the pendency of the application to the Commissioner of Patents and Trademarks and persons determined by the Commissioner to be entitled thereto upon request. Upon allowance of any claims in the application, the Applicant(s) will maintain and will make this deposit available to the public pursuant to the Budapest Treaty.

The present invention also provides F₁ hybrid corn plants exhibiting both a bm3 and fl2 genotype and a brown-midrib and floury-endosperm phenotype. The physical characteristics of an exemplary corn hybrid exhibiting both the brown-midrib and a floury-endosperm phenotype compared to a normal grain corn hybrid are set forth in Table 2.

TABLE 2
Phenotype of the Homozygous-Recessive bmr/fl2 Genotype
Compared to the Dominant BMR/FL Genotype

| | bm3/bm3, fl2/fl2 | BMR/BMR, |
|---|---|---|
| | Hybrid 09SMA31BF × | FL/FL |
| Character | 09IAA63BF^{a} | Hybrid 2W587 |
| leaf mid-rib color V4 to V6^{b} | reddish-brown | green |
| stalk color | reddish-brown | green |
| NDFD (%)^{c} | 70.2 | 55.6 |
| grain light transmision | opaque | translucent |

^{a}Hybrid made by pollinating inbred 09SMA31BF with pollen from inbred 09IAA63BF.
^{b}Corn V4 to V6 growth stages have four to six leaves.
^{c}Percentage of neutral detergent fiber that is digestible.

Only the preferred embodiment of the invention and but a few examples of its versatility are shown and described in the present disclosure. It is to be understood that the present invention is capable of use in various other combinations and environments and is capable of changes or modifications within the scope of the inventive concept as expressed herein.

We claim:

1. Corn seed comprising a homozygous brown-midrib-3 (bm3) and a homozygous floury-2 (fl2) genotype.

2. A corn plant comprising a brown-midrib and a floury-endosperm phenotype produced by growing the seed of claim 1.

3. A part of the corn plant of claim 2, selected from the group consisting of an intact plant cell, a plant protoplast, an embryo, a pollen, an ovule, a flower, a kernel, a seed, an ear, a cob, a leaf, a husk, a stalk, a root, a root tip, a brace root, a lateral tassel branch, an anther, a tassel, a glume, a tiller and a silk.

4. The corn plant according to claim 2, wherein the brown-midrib phenotype is a result of a homozygous recessive bm3 genotype.

5. The corn plant according to claim 2, wherein the floury-endosperm phenotype is a result of a homozygous recessive fl2 genotype.

6. Seed of a corn inbred line comprising a homozygous bm3 and fl2 genotype, or a part thereof.

7. An inbred corn plant comprising a homozygous bm3 and fl2 genotype produced by growing the seed of claim 6.

8. A part of the corn plant of claim 7, selected from the group consisting of an intact plant cell, a plant protoplast, an embryo, a pollen, an ovule, a flower, a kernel, a seed, an ear, a cob, a leaf, a husk, a stalk, a root, a root tip, a brace root, a lateral tassel branch, an anther, a tassel, a glume, a tiller and a silk.

9. Seed of corn inbred line designated 09SMA31BF, or a part thereof, representative seed of the line having been deposited under ATCC Accession No. ______.

10. A method for producing inbred corn seed comprising a homozygous bm3 and fl2 genotype, comprising:
(a) planting inbred corn seeds comprising a homozygous bm3 and fl2 genotype in proximity to itself;
(b) growing plants from the seed under pollinating conditions; and,
(c) harvesting resultant seed.

11. A corn plant comprising a homozygous bm3 and fl2 genotype produced by growing the harvested, resultant seed of claim 10.

12. Pollen of the plant of claim 7.

13. An ovule of the plant of claim 7.

14. A method for producing a hybrid corn seed comprising a homozygous bm3 and fl2 genotype, the method comprising the steps of:
(a) planting in pollinating proximity seeds of a first and a second inbred parent corn plants, wherein the first inbred corn plant and the second inbred corn plant both comprise a homozygous bm3 and fl2 genotype;
(b) cultivating the seeds of the first and the second inbred corn plants into plants that bear flowers;
(c) controlling the male fertility of the first or the second inbred corn plant to produce a male sterile corn plant;
(d) allowing cross-pollination to occur between the first and second inbred corn plants; and,
(e) harvesting seeds produced on the male sterile corn plant.

15. A hybrid corn seed produced by the method of claim 14.

16. A hybrid corn plant, or parts thereof, producing by growing the hybrid corn seed of claim 15.

17. A method of introducing a brown-midrib trait and floury-endosperm trait into a corn inbred line comprising:
(a) crossing recurrent inbred corn plants with donor plants of another corn line that comprise a desired brown-midrib trait and a desired floury-endosperm trait to produce F₁ progeny plants;
(b) crossing F₁ progeny plants with the recurrent inbred corn plants to produce backcross (BC 1) progeny plants, which are then self pollinated to produce BC1S1 plants;
(c) selecting for BC1S1 progeny seeds and plants that respectively comprise the desired floury-endosperm and brown-midrib traits, and physiological and morphological characteristics of the recurrent corn inbred line; and crossing the selected BC1S1 plants with the recurrent inbred corn plants to produce the BC2S1 progeny plants, which are then self pollinated to produce BC2S2 plants;
(e) performing steps (b) and (c) one or more times in succession to produce the selected or higher backcross progeny plants that comprise the desired brown-midrib trait and floury-endosperm traits and all of the physiological and morphological characteristics of recurrent corn inbred line as determined at the 5% significance level when grown in the same environmental conditions.

18. The method of claim 17, further comprising using genetic markers to identify the bm3 and fl2 alleles and compare a genetic complement of a progeny plant with a genetic complement of the recurrent corn inbred line.

19. A method for producing a derived corn plant, comprising:
(a) crossing an inbred corn line comprising a bm3 and fl2 genotype with a second corn plant to yield progeny corn seed; and
(b) growing said progeny corn seed under plant growth conditions to yield the derived corn plant.

20. A derived corn plant, or parts thereof, produced by the method of claim 19.

21. The method of claim 19, further comprising:
(c) crossing the derived corn plant with itself or another corn plant to yield additional derived progeny corn seed;
(d) growing the progeny corn seed of step c) under plant growth conditions to yield additional derived corn plants; and
(e) repeating the crossing and growing steps of c) and d) from 0 to 7 times to generate further derived corn plants.
